(19) United States
(12) Patent Application Publication
(10) Pub. No.: US 2016/0023984 A1
(43) Pub. Date: Jan. 28, 2016

(54) MANUFACTURING METHOD OF ESTER COMPOUND

(71) Applicants: Mitsunobu MORITA, Shizuoka (JP); Soh NOGUCHI, Kanagawa (JP); Daisuke MIKI, Kanagawa (JP)

(72) Inventors: Mitsunobu MORITA, Shizuoka (JP); Soh NOGUCHI, Kanagawa (JP); Daisuke MIKI, Kanagawa (JP)

(21) Appl. No.: 14/741,914

(22) Filed: Jun. 17, 2015

(30) Foreign Application Priority Data
Jul. 24, 2014 (JP) 2014-150660

(51) Int. Cl. C07C 67/03

(52) U.S. Cl. CPC C07C 67/03

(57) ABSTRACT

A manufacturing method of an ester compound includes reacting, in a nonpolar solvent, a compound including, in one molecule of the compound, an ester structure and an alcoholic hydroxyl group with a compound that abstracts a proton from the alcoholic hydroxyl group.

### CROSS-REFERENCE TO RELATED APPLICATION

This patent application is based on and claims priority pursuant to 35 U.S.C. §119 from Japanese Patent Application No. 2014-150660, filed on Jul. 24, 2014 in the Japan Patent Office, which is hereby incorporated by reference herein in its entirety.

### BACKGROUND

1. Technical Field

Exemplary embodiments of the present disclosure generally relate to a manufacturing method of an ester compound.

2. Description of the Related Art

Many publications describe methods for esterification of an alcoholic hydroxyl group. Among such methods, a dehydration reaction with a carboxylic acid and an alcohol is the most common method of synthesizing an ester, and is often conducted in the presence of an acid (i.e., acid catalyst) such as sulfuric acid and hydrochloric acid. Esterification with the dehydration reaction is an equilibrium reaction. Thus, in order to further esterification, a scheme to remove water generated by the dehydration reaction from a reaction system, employing excess of one of the reaction materials (e.g., carboxylic acid or alcohol), or the like are implemented.

In a case of a reaction of an alcohol compound and a carboxylic acid compound, a method to remove water generated by the reaction may be employing an aromatic hydrocarbon based solvent such as toluene and benzene in a process of heat treatment, in the presence of sulfuric acid, and forming an azeotrope with the aromatic hydrocarbon based solvent. In the above-described case, heating is often conducted for a long period with respect to the reaction and heating to an azeotropic point of the aromatic hydrocarbon based solvent and water is necessary. Further, to isolate an ester compound from the reaction, in addition to a neutralizing process of the acid, there is a need for a refining process such as distillation. In a case in which a by-product is generated due to conducting the reaction under heating conditions for the long period, there is a need for an additional refining process.

Regarding the above-described case of esterification with the dehydration reaction employing the acid catalyst, when the alcohol compound is a primary alcohol, the reaction easily progresses. When the alcohol compound is a secondary alcohol or a tertiary alcohol, the reaction does not easily progress and a need for a particular catalyst or strict reaction conditions occur.

There are various methods to esterify the alcoholic hydroxyl group. However, there are issues such as a need for heating conditions or cooling conditions with respect to a reaction, and difficulty with respect to progressing a reaction depending upon a type of an alcohol that is employed in the reaction. Further, there is a need to remove a catalyst, and a refining process such as distillation is necessary. Thus, there is a demand for a simple manufacturing method.

### SUMMARY

In view of the foregoing, in an aspect of this disclosure, there is provided a novel manufacturing method of an ester compound including reacting, in a nonpolar solvent, a compound including, in one molecule of the compound, an ester structure and an alcoholic hydroxyl group with a compound that abstracts a proton from the alcoholic hydroxyl group.

These and other aspects, features, and advantages will be more fully apparent from the following detailed description of illustrative embodiments, the accompanying drawings, and associated claims.

### DETAILED DESCRIPTION

Hereinafter, exemplary embodiments of the present invention are described in detail with reference to the drawings. However, the present invention is not limited to the exemplary embodiments described below, but may be modified and improved within the scope of the present disclosure.

In describing embodiments illustrated in the drawings, specific terminology is employed for the sake of clarity. However, the disclosure of this patent specification is not intended to be limited to the specific terminology so selected and it is to be understood that each specific element includes all technical equivalents that have the same function, operate in a similar manner, and achieve similar results.

There is provided a novel manufacturing method of an ester compound in which the ester compound is derived from a compound (hereinafter may be referred to as compound A) that includes, in one molecule of the compound, an ester structure and an alcoholic hydroxyl group, and simple esterification of the alcoholic hydroxyl group.

In the manufacturing method according to an embodiment of the present invention, esterification of the compound A progresses as follows. The compound A is reacted with a compound that abstracts a proton from the alcoholic hydroxyl group of the compound A. Accordingly, an alkoxy anion is generated in the reacted compound A. Then, the generated alkoxy anion of the reacted compound A reacts with an ester structure of another molecule of the compound A.

For example, the following reaction formula (1) is a case of reacting glycerin dimethacrylate with sodium hydride at room temperature. (In reaction formula (1), R3 represents a hydrogen atom or a methyl group)

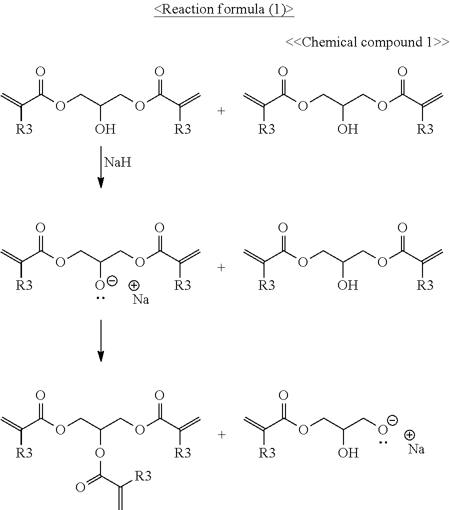

Esterification in the manufacturing method of the present invention is conducted in a nonpolar solvent. For example, when the above-described reaction formula (1) is conducted in a nonpolar solvent, a sodium salt of glycerin monoacrylate or glycerin monomethacrylate, generated as a by-product of the reaction, precipitates and does not dissolve in the nonpolar solvent. Accordingly, the by-product can be removed by filtration. Thus, with the manufacturing method of the present invention, a target ester compound (i.e., in the above-described reaction formula (1), glycerin triacrylate or glycerin trimethacrylate) can be easily obtained by concentrating a filtrate and removing the nonpolar solvent.

More specifically, with the manufacturing method of the present invention, esterification is possible with simple procedures of filtration and concentration at room temperature.

Specific examples of the compound A are shown in the following <<Chemical compound 2>>. With the manufacturing method of the present invention, an alcoholic hydroxyl group (—OH) in the following compounds can be changed to an ester structure (—OOCR1). In the following structural formulas, R1 represents an alkyl group, a vinyl group that may be replaced with an alkyl group, or an allyl group that may be replaced with an alkyl group.

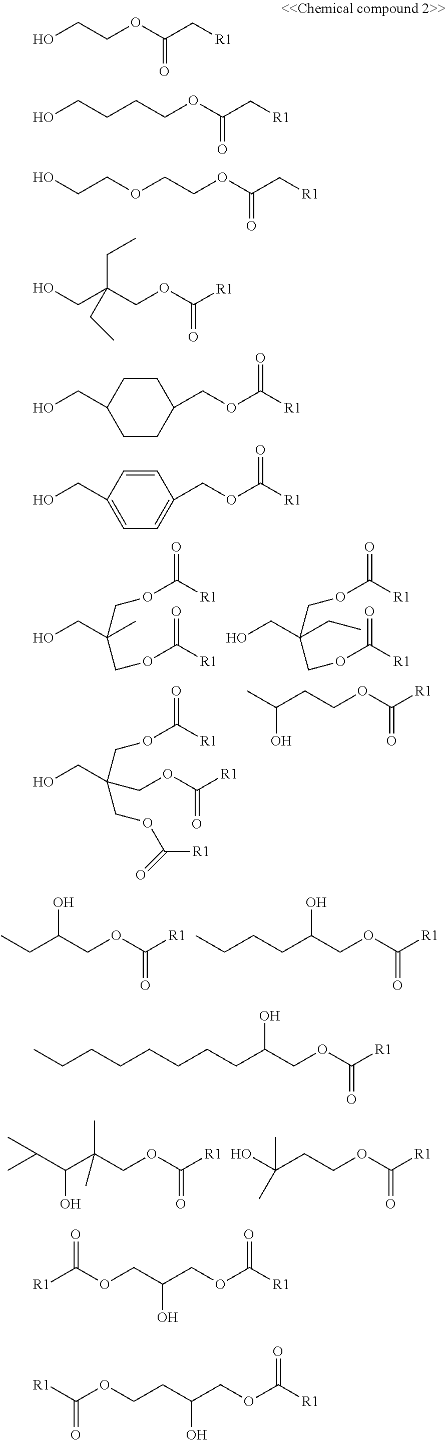

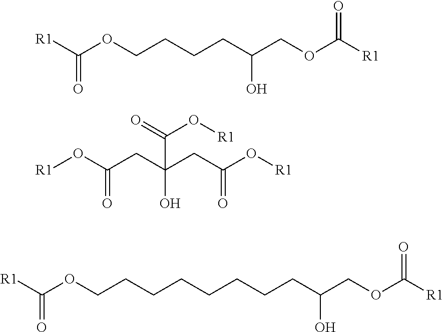

Specific examples of the compound that abstracts the proton from the alcoholic hydroxyl group include, but are not limited to, alkali metals such as lithium, sodium, and potassium; metal hydrides such as lithium hydride, potassium hydride, calcium hydride, sodium hydride, sodium borohydride, and lithium aluminium hydride; and alkoxide compounds such as sodium methoxide, sodium ethoxide, and sodium tertiary butoxide. Among the above-described examples of the compound that abstracts the proton from the alcoholic hydroxyl group, handling of sodium hydride is easy and is preferable.

Specific examples of the nonpolar solvent employed in a reaction include, but are not limited to, aromatic hydrocarbons such as benzene, toluene, and xylene; aliphatic hydrocarbons such as pentane, hexane, and heptane; and aliphatic cyclic hydrocarbons such as cyclopentane and cyclohexane. Among the above-described nonpolar solvents, toluene and hexane are preferable in view of cost. Hexane with a low boiling point is particularly preferable.

### EXAMPLES

Further understanding can be obtained by reference to specific examples and comparative examples, which are provided hereinafter. However, it is to be understood that the embodiments of the present invention are not limited to the following examples. It is to be noted that in the following examples, “%” refer to “% by mass”. Further, in the following examples, ¹H-NMR spectra are measured with a ¹H-NMR spectrometer (500 MHz) (from JEOL Ltd.), and IR spectra are measured with a FT-IR Spectrum GX system (from Perkin Elmer Co., Ltd.).

### Example 1

Sodium hydride (3.05 g, 70 mmol) 55% in liquid paraffin was subjected to cleaning with toluene, and the liquid paraffin was removed. After removing the liquid paraffin, 150 mL of toluene was added to sodium hydride at room temperature and stirred. Then, glycerin-1,3-dimethacrylate (15.98 g, 70 mmol) was slowly dripped into the above-described mixture of sodium hydride and toluene at room temperature. Then, precipitated 12.0 g of light yellow-white solid precipitate was removed by filtration. Next, by employing a rotary evaporator, toluene was removed from the filtrate (i.e., the mixture of sodium hydride, toluene, and glycerin-1,3-dimethacrylate after filtration). Accordingly, glycerin trimethacrylate as shown in the following <<Chemical compound 3>> was obtained. Obtained amount was 5.1 g (yield is 25%).

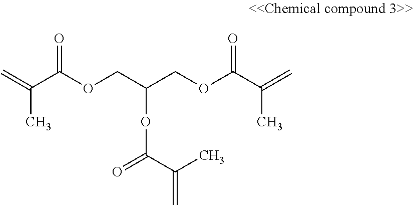 [SMILES: C=C(C)C(=O)OCC(COC(=O)C(=C)C)OC(=O)C(=C)C]

¹H-NMR (CDCl₃): δ1.94 (s, 9H), 4.30-4.36 (m, 2H), 4.40-4.44 (m, 2H), 5.42-5.46 (m, 1H), 5.59-5.62 (m, 3H), 6.10-6.14 (m, 3H)

IR (NaCl): 2960, 2929, 1725, 1638, 1454, 1404, 1378, 1324, 1294, 1158, 1097, 1064, 1011, 943, 855, 813, 652 cm⁻¹

### Example 2

Sodium hydride (3.05 g, 70 mmol) 55% in liquid paraffin was subjected to cleaning with hexane, and the liquid paraffin was removed. After removing the liquid paraffin, 150 mL of hexane was added to sodium hydride at room temperature and stirred. Then, glycerin-1,3-dimethacrylate (15.98 g, 70 mmol) was slowly dripped into the above-described mixture of sodium hydride and hexane at room temperature. Then, precipitated 10.8 g of white solid precipitate was removed by filtration. Next, by employing a rotary evaporator, hexane was removed from the filtrate (i.e., the mixture of sodium hydride, hexane, and glycerin-1,3-dimethacrylate after filtration). Accordingly, glycerin trimethacrylate as shown in <<Chemical compound 3>> was obtained. Obtained amount was 6.6 g (yield is 32%). Measurement data of ¹H-NMR and IR was the same as example 1.

### Comparative Example 1

Glycerin (4.6 g, 50 mmol) and triethylamine (18.21 g, 180 mmol) was added to 140 mL of dehydrated dichloromethane, and was cooled to approximately −15° C. in an ice bath. Then, methacrylic acid chloride (18.82 g, 180 mmol) was slowly dripped into the above-described mixture of glycerin, triethylamine, and dehydrated dichloromethane. A temperature of a reaction system of the above-described mixture and methacrylic acid chloride was maintained at approximately −5° C. The reaction mixture (i.e., the above-described mixture and methacrylic acid chloride) was stirred for fifteen minutes at −5° C. Then, the reaction mixture was stirred for approximately one hour at room temperature. Then, the reaction mixture was subjected to filtration, and precipitate in the reaction mixture was removed. The filtrate (i.e., the reaction mixture after filtration) was cleaned with water, a saturated sodium bicarbonate solution, and a saturated saline solution. Then, the cleaned filtrate was dried with sodium sulfate, and was concentrated employing a rotary evaporator. Accordingly, 17.8 g of a brown color solution was obtained.

Next, the obtained brown color solution was subjected to column chromatography (eluent: hexane/ethyl acetate mixture solvent) employing silica gel (WAKOGEL C-300). Accordingly, glycerin trimethacrylate as shown in <<Chemical compound 3>> was obtained. Obtained amount was 2.6 g (yield is 18%). Measurement data of ¹H-NMR and IR was the same as example 1.

### Comparative Example 2

Glycerin (2.76 g, 30 mmol) and methacrylic acid (10.33 g, 120 mmol) was added to 130 mL of toluene, and then 0.1 g of concentrated sulfuric acid was further added. Then, the above-described mixture (i.e., glycerin, methacrylic acid, toluene, and concentrated sulfuric acid) was stirred at room temperature. After stirring, the above-described mixture was heated in an oil bath and refluxed for eight hours. Then, the reaction mixture (i.e., the above-described mixture after refluxing) was cleaned with water, a saturated sodium bicarbonate solution, and a saturated saline solution. Next, the cleaned reaction mixture was dried with sodium sulfate. After drying, toluene was removed from the cleaned reaction mixture employing a rotary evaporator. Accordingly, glycerin-1,3-dimethacrylate as shown in the following <<Chemical compound 4>> was obtained. Obtained amount was 4.4 g.

A target ester compound of glycerin trimethacrylate was not obtained.

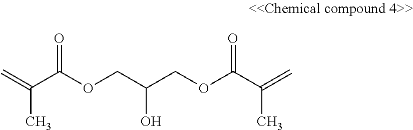 [SMILES: C=C(C)C(=O)OCC(O)COC(=O)C(=C)C]

¹H-NMR (CDCl₃): δ1.96 (s, 6H), 2.84 (bs, 1H), 4.17-4.22 (m, 1H), 4.23-4.32 (m, 4H), 5.61-5.63 (m, 2H), 6.14-6.16 (m, 2H)

IR (NaCl): 3490, 2961, 2930, 1722, 1636, 1455, 1407, 1377, 1321, 1297, 1165, 1046, 1013, 946, 899, 815, 734, 652 cm⁻¹

In view of the foregoing, with the manufacturing method according to the present invention, esterification of the alcoholic hydroxyl group of the compound A is possible with a very simple method. With the manufacturing method of the present invention, esterification of the alcoholic hydroxyl group of one molecule of the compound A is obtained from two molecules of the compound A. On a basis of raw materials, a reaction yield is maximum 50%. However, the target ester compound is obtained by simply mixing raw material compounds at room temperature, conducting filtration after reaction of the mixed raw material compounds, and concentrating the filtrate (i.e., the mixed raw material compounds after filtration). The manufacturing method is very simple and useful.

The following are descriptions of aspects of the above-described exemplary examples of the present invention.
[Aspect 1]

A manufacturing method of an ester compound that includes reacting, in a nonpolar solvent, a compound A including, in one molecule of the compound A, an ester structure and an alcoholic hydroxyl group with a compound that abstracts a proton from the alcoholic hydroxyl group.
[Aspect 2]

The manufacturing method of the ester compound of aspect 1 in which the compound A is expressed by a general formula 1-1 or a general formula 1-2.

C(OH)r(R2)q[X—O—C(═O)—R1]p<General formula 1-1>

C(OH)r(R2)q[X—C(═O)—O—R1]p<General formula 1-2>

(In general formulas 1-1 and 1-2, X represents a straight chain or a branched chain alkylene group; R1 represents an alkyl group, a vinyl group that may be replaced with an alkyl group, or an allyl group that may be replaced with an alkyl group; R2 represents a hydrogen atom or an alkyl group; p represents an integer of 1 to 3; q represents an integer of 0 to 2; r represents an integer of 1 to 3; and p+q+r=4)
[Aspect 3]

The manufacturing method of the ester compound of aspect 1 in which the alcoholic hydroxyl group is a secondary alcohol.
[Aspect 4]

The manufacturing method of the ester compound of aspect 1 in which the ester structure is an acrylic acid ester or a methacrylic acid ester.
[Aspect 5]

The manufacturing method of the ester compound of aspect 4 in which the compound A is expressed by a general formula 2.

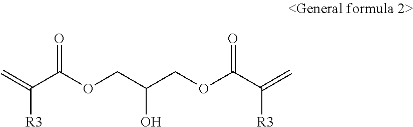

(In general formula 2, R3 represents a hydrogen atom or a methyl group)
[Aspect 6]

The manufacturing method of the ester compound of aspect 1 in which the nonpolar solvent is a hydrocarbon based solvent.
[Aspect 7]

The manufacturing method of the ester compound of aspect 6 in which the hydrocarbon based solvent is hexane.

[Aspect 8]

The manufacturing method of the ester compound of aspect 1 in which the compound that abstracts the proton from the alcoholic hydroxyl group is sodium hydride.

What is claimed is:

1. A manufacturing method of an ester compound, comprising:
reacting, in a nonpolar solvent, a compound including, in one molecule of the compound, an ester structure and an alcoholic hydroxyl group with a compound that abstracts a proton from the alcoholic hydroxyl group.

2. The manufacturing method of the ester compound of claim 1, wherein the compound including, in one molecule of the compound, the ester structure and the alcoholic hydroxyl group is expressed by a general formula 1-1 or a general formula 1-2,
C(OH)r(R2)q[X—O—C(═O)—R1]p< General formula 1-1>
C(OH)r(R2)q[X—C(═O)—O—R1]p< General formula 1-2>
wherein, with respect to the general formulas 1-1 and 1-2, X represents a straight chain or a branched chain alkylene group, R1 represents an alkyl group, a vinyl group that may be replaced with an alkyl group, or an allyl group that may be replaced with an alkyl group, R2 represents a hydrogen atom or an alkyl group, p represents an integer of 1 to 3, q represents an integer of 0 to 2, r represents an integer of 1 to 3, and p+q+r=4.

3. The manufacturing method of the ester compound of claim 1, wherein the alcoholic hydroxyl group is a secondary alcohol.

4. The manufacturing method of the ester compound of claim 1, wherein the ester structure is an acrylic acid ester or a methacrylic acid ester.

5. The manufacturing method of the ester compound of claim 4, wherein the compound including, in one molecule of the compound, the ester structure and the alcoholic hydroxyl group is expressed by a general formula 2, 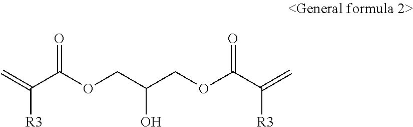
wherein R3 in general formula 2 represents a hydrogen atom or a methyl group.

6. The manufacturing method of the ester compound of claim 1, wherein the nonpolar solvent is a hydrocarbon based solvent.

7. The manufacturing method of the ester compound of claim 6, wherein the hydrocarbon based solvent is hexane.

8. The manufacturing method of the ester compound of claim 1, wherein the compound that abstracts the proton from the alcoholic hydroxyl group is sodium hydride.
